(19) United States
(12) Patent Application Publication
(10) Pub. No.: US 2008/0217562 A1
(43) Pub. Date: Sep. 11, 2008

(54) METHOD FOR REFORMING CARBONACEOUS MATERIALS

(75) Inventor: Takao SAITO, Nagoya-City (JP)

Correspondence Address: BURR & BROWN, PO BOX 7068, SYRACUSE, NY, 13261-7068, US

(73) Assignee: NGK Insulators, Ltd., Nagoya-City (JP)

(21) Appl. No.: 12/042,525

(22) Filed: Mar. 5, 2008

Related U.S. Application Data
(60) Provisional application No. 60/893,662, filed on Mar. 8, 2007.

(30) Foreign Application Priority Data
Mar. 6, 2007 (JP) 2007-055232

(51) Int. Cl. A61N 5/00

(52) U.S. Cl. 250/492.3

(57) ABSTRACT

A carbonaceous material containing 30 atm % or more of carbon atom is reformed. The reforming is carried out by applying a DC pulse voltage to an electrode set within a chamber to generate an electron beam, and by then irradiating a surface of the carbonaceous material with the electron beam. The DC pulse voltage has a duty ratio of pulse duration per pulse of 0.05 to 5.0%, an input energy of 0.01 J/cm² or less and a pulse half-value width of 10 to 900 nsec.

### BACKGROUND OF THE INVENTION

1. Field of the Invention

The present invention relates to a method for reforming a carbonaceous material.

2. Description of the Related Art

A surface treatment method for a metal mold by electron beam irradiation is described in Japanese Patent Publication No. 2004-1086A, wherein pulse width of pulse voltage is 1.0 μs (1000 nsec) or more and energy density is 1 J/cm² or more. Particularly the surface roughness of the metal mold is reduced at an energy density of pulse voltage of 1 to 4 J/cm² and minimized at 6 to 7 J/cm². This shows that the treatment is a processing for smoothing a metal surface by minutely melting the metal surface.

An electron beam apparatus for reforming a surface of a metallic denture is disclosed in Japanese Patent Publication No. 2003-111778A, so that a magnetic field generating means is provided at an electron generation part to generate a magnetic field. Pulse width of pulse voltage is 0.5 to 10 μs, and electron irradiation energy density is 0.1 J/cm² or more (refer to claims). However, the irradiation energy density is particularly recommended to be 2 J/cm² or more based on FIG. 3, and this description apparently describes a method of minutely melting a metal surface.

Further, it is described in Japanese Patent Publication No. 2006-187799A that an electron beam irradiation device is disposed in a magnetic field by confining an electron gun by excitation of a solenoid. In this technique, also, the irradiation energy density is basically set to 2 J/cm² or more (FIGS. 10 and 12), so that smoothing of a metal surface by locally melting the metal surface followed by resolidifying is described.

Further, treatment of a low-k dielectric film by ion implantation is described in Japanese Patent publication No. 2006-526899A.

### SUMMARY OF THE INVENTION

Japanese Patent Publication Nos. 2004-1086A, 2003-111778A and 2006-187799A intend to smooth a metal surface by locally melting the metal surface followed by resolidifying. An energy with pulse width of 1.0 μs (1000 nsec) or more and electronic energy density of 0.1 J/cm² or more is needed therefor, because the metal surface must be heated to the melting point for melting.

An amorphous carbon film containing carbon elements, which has been used as a wear-resistant film, is requested to be further improved in wear resistance and reduced in coefficient of friction. When the metal surface smoothing methods as described above are applied thereto, however, purpose-based processing is difficult because the minute shape of the surface is seriously affected.

An organic thin film such as a low-k film or a photoresist is known to be extremely fragile and has a low melting point. The ion implantation method as described in Japanese Patent publication No. 2006-526899A causes nanometer- or micron-order deformation of shape of the organic thin film, since etching treatment is carried out simultaneously with ion implantation.

In Japanese Patent Application No. 2006-12264 (Publication No. 2007-194110A), it is described that in generation of discharge plasma by applying a pulse voltage to treatment gas, plasma with high electron density and low electron temperature can be generated by controlling the duty ratio of the pulse voltage to 0.001% or more and 8.0% or less. However, it is not described that such plasma can be used for reforming a carbonaceous material surface.

The present invention thus provides a method for reforming a carbonaceous material while suppressing change in minute shape of surface of the carbonaceous material.

The present invention provides a method for reforming a carbonaceous material comprising 30 atm % or more of carbon atoms; said method comprising the steps of

applying a DC pulse voltage to electrodes set within a chamber to generate an electron beam; and

irradiating a surface of the carbonaceous material with the electron beam. In this case, the duty ratio of pulse duration per pulse of the DC pulse voltage is set to 0.005 to 5.0%, input energy is set to 0.01 J/cm² or less, and the pulse half-value width of the DC pulse voltage is set to 10 to 900 nsec.

The present inventors found that physical properties of a carbonaceous material can be reformed without greatly changing the minute shape of a surface of the carbonaceous material, by irradiating the surface of the carbonaceous material with an electron beam having remarkably small duty ratio, input energy and pulse width without giving a magnetic field. The present invention is thus made.

### BRIEF DESCRIPTION OF THE DRAWINGS

FIG. 1 is a view for illustrating the half-value width of pulse in the present invention.

FIG. 2 is a schematic view showing one example of an apparatus usable for carrying out the present invention.

FIG. 3 is a schematic view showing another example of an apparatus usable for carrying out the present invention.

### BEST MODES FOR CARRYING OUT THE INVENTION

The carbonaceous material in the present invention means a material having a ratio of carbon atoms of 30 atm % or more. The ratio of carbon atoms in the material is determined by the following method.

The atomic content can be determined by Auger electron spectroscopy (AES) analysis.

Concretely the carbonaceous material of the present invention includes a carbon substantially composed of carbon atoms or an organic resin mainly composed of carbon and hydrogen atoms. The carbonaceous material is preferably a low-dielectric constant material. The low-dielectric constant material means a material having a specific permittivity of 2.8 or less.

As the carbon, amorphous carbon, diamondlike carbon and graphite are preferred, and amorphous carbon and diamondlike carbon are particularly preferred. As the low-dielectric constant material, a photoresist and a low-k, that is an interlayer insulating film for semiconductor element, are particularly preferred. As the organic resin, polyethylene, polypropylene, polystyrene, polycarbonate, polyethylene terephthalate, polytetrafluoroethylene and acrylic resins are preferred.

The form of the carbonaceous material that is a treatment object is not particularly limited, and the carbonaceous material may have a sheet-like form, a film-like form or the like. The treatment method of the present invention can easily applied to treatment of substrates having various shapes.

The reforming of the carbonaceous material means to change physical properties thereof so as to be adapted to an intended use without being limited to only a specific property. However, for example, reduction in coefficient of friction, increase in surface hardness, and improvement in durability (for example, plasma resistance) of the carbonaceous material can be attained thereby.

In the present invention, the electron beam is generated by applying a DC pulse voltage to electrodes set within a chamber. The duty ratio of pulse duration per pulse of the DC pulse voltage is set to 0.005 to 5.0%. The shape change of the surface of the carbonaceous material can be minimized by setting the duty ratio to 5.0% or less. From this point of view, it is further preferable to set the duty ratio to 3% or less. It is practically difficult to set the lower limit thereof to less than 0.005%.

The duty ratio of the pulse voltage is represented by the following equation.

Duty ratio (%)=(Sum of ON time of pulse/Pulse period)×100

The “ON time of pulse” means the time from rising start of pulse to trailing end of pulse.

The “Sum of ON time of pulse” is a total value of ON times of all pulses contained in one period.

In application of positive pulse with ON time of 1 μsec and a period of 1000 μsec, for example, the duty ratio is (1 μsec/1000 μsec)×100=0.1%.

When positive pulse and negative pulse are alternately contained in one period, the total value of ON time of the positive pulse and ON time of the negative pulse is divided by one period. In application of positive pulse with ON time of 1 μsec and negative pulse with 2 μsec in a period of 1000 μsec, the duty ratio is ((1 μsec+2 μsec)/1000 μsec)×100=0.3%.

In the present invention, the input energy for generating the electron beam is set to 0.01 J/cm² or less. According to this, the carbonaceous material can be successfully reformed while suppressing the change in the surface shape thereof. If the input energy is large, roughing, deformation and irregularities of the carbonaceous material surface become serious. Even if the electron beam is generated with the input energy of 0.01 J/cm² or less and irradiated to a metal surface without a magnetic field, no local melting or resolidifying of the metal surface was substantially caused and the reforming effect was not particularly observed.

The input energy is set further preferably to 0.001 J/cm² or less. Although the lower limit of the input energy is not particularly defined, it is preferable to practically set the lower limit to 0.0000001 J/cm² or more.

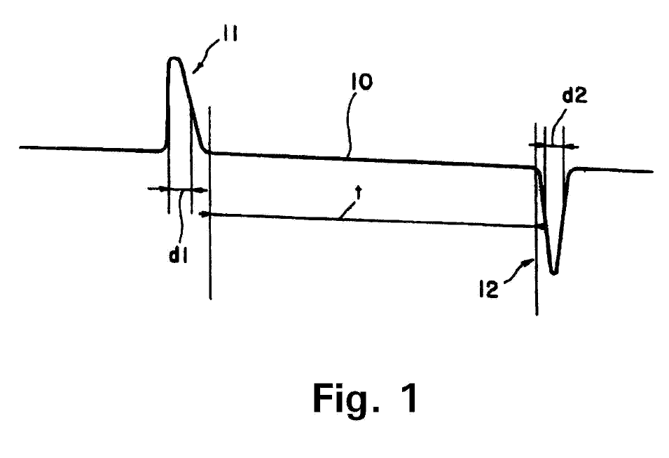

In the present invention, the pulse half-value width of the DC pulse voltage is set to 10 to 900 nsec. The pulse half-value width means the interval between start voltage and end voltage where the maximum voltage of DC pulse is halved. In a pulse voltage waveform 10 as shown in FIG. 1, for example, positive pulse 11 and negative pulse 12 are alternately applied at a fixed period. In the drawing, denoted at d1 is the half-value width of the positive pulse 11, and d2 is the half-value width of the negative pulse 12.

The carbonaceous material can be reformed while suppressing the change in surface shape thereof by setting the pulse half-value width of the DC pulse voltage to 900 nsec or less. From this point of view, it is further preferable to set the pulse half-value width of the DC pulse voltage to 800 nsec or less.

In a preferred embodiment, the DC pulse voltage has a pulse period of 0.01 to 100 kHz, and a pulse voltage of ±0.1 to ±30 kV.

In another preferred embodiment, the discharge plasma generated by using the electron beam is glow discharge plasma. However, hollow cathode discharge, streamer discharge or arc discharge may be adapted.

Although the pressure of the treatment gas is not limited, the internal pressure of the chamber can be set to 0.1 to 1000 Pa. However, the present invention is most effective for a process for generating discharge plasma in a low-pressure condition. From this point of view, the pressure of the treatment gas is set preferably to 100 Pa or less and, further preferably to 50 Pa or less.

In the present invention, it is particularly preferable to apply at least either of positive pulse and negative pulse, and a thin film, for example, can be formed thereby with high efficiency. In this case, each application pattern of the positive pulse and the negative pulse is not particularly limited. Multiple continuous applications of the positive pulses or multiple continuous applications of the negative pulses can be also performed.

The magnitude of the positive pulse 11 is not particularly limited. However, for example, the field intensity between opposed electrodes is set preferably to 0.01 to 100 kV/cm and, further preferably to 0.1 to 50 kV/cm.

The magnitude of the negative pulse 12 is not particularly limited. However, for example, the field intensity between opposed electrodes is set preferably to −0.1 to −100 kV/cm and, further preferably to −0.1 to −50 kV/cm.

In the present invention, the electron beam is generated in the space between the opposed electrodes. Although a dielectric body may be set on at least one of the opposed electrodes, the metallic electrode may be exposed. As the opposed electrodes, for example, parallel plate type, cylindrical opposed plate type, spherical opposed plate type, hyperbolic opposed plate type, and coaxial cylindrical structures can be given.

As the solid dielectric which covers one or both of the opposed electrodes, for example, a plastic such as polytetrafluoroethylene or polyethylene terephthalate, and a metallic oxide such as glass, silicon dioxide, aluminum oxide, aluminum nitride, zirconium dioxide or titanium dioxide, and a composite oxide such as barium titanate can be given.

The dielectric body preferably has a thickness of 0.05 to 4 mm. The distance between the opposed electrodes is not particularly limited, but is set preferably to 1 to 500 mm. Examples of the material of the substrate include plastics such as polyethylene, polypropylene, polystyrene, polycarbonate, polyethylene terephthalate, polyphenylene sulfite, polyether ether ketone, polytetrafluoroethylene or acrylic resin, glass, ceramics and metal. The shape of the dielectric body is not particularly limited, and various three-dimensional shapes such as sheets and films can be adapted.

In the present invention, the electron beam is generated by applying the pulse voltage between the opposed electrodes. Each pulse waveform of positive pulse and negative pulse is not particularly limited, and may be any one of impulse type, square wave type (rectangular wave type) and modulated type. The DC bias voltage can be applied simultaneously.

The electrode set in vacuum for releasing the electron beam preferably has a flat plate shape of φ100 mm or more or a wire-like shape of φ5 mm or less.

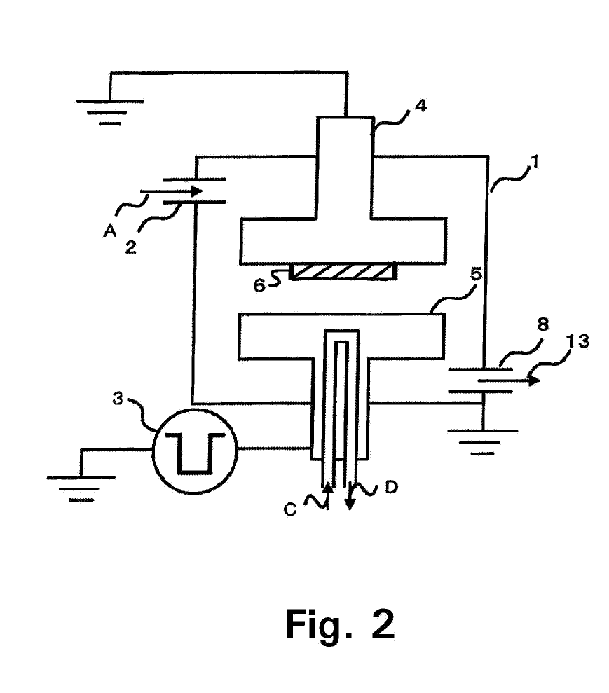

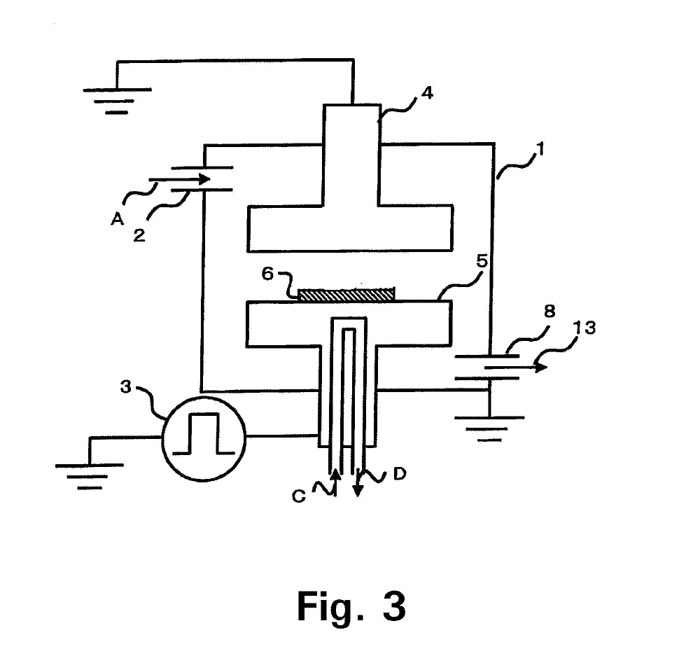

FIGS. 2 and 3 are views schematically showing apparatuses usable in the present invention. Generation of the discharge plasma is carried out within a chamber 1. In the example of FIG. 3, a substrate 6 is set on a lower electrode 5 to be opposed to an upper electrode 4, and the discharge plasma using electron beam is generated in the space between the substrate 6 and the upper electrode 4. In the example of FIG. 2, the substrate 6 is set on the upper electrode 4. The plasma is generated by supplying a raw material gas through a gas supply port 2 of the chamber 1 as shown by arrow A, and applying a pulse voltage including positive pulse and negative pulse between the electrodes from a power source 3 using an electrostatic induction thyrister device. The used gas is discharged through a discharge port 8 as shown by arrow B. A distribution passage for refrigerant is formed within the lower electrode 5, and refrigerant is distributed into the distribution passage as shown by arrows C and D. The temperature of the substrate 6 is controlled thereby to a predetermined temperature, for example, to 20 to 800° C.

The raw material gases may be supplied into the chamber 1 after being thoroughly mixed. When the raw material gas contains two or more kinds of gases and a diluent gas, the respective gases may be supplied into the chamber 1 through independent supply ports.

The pulse voltage may be applied by a steep pulse generating power source. Examples of such a power source include a power source using static induction thyrister device which needs no magnetic compression mechanism, and a power source using thyratron, gap switch, IGBT element, MOF-FET device, or electrostatic thyrister element which are provided with a magnetic compression mechanism.

As the treatment gas, hydrogen, oxygen-based gas, rare gas, fluoride-based gas, and chloride-based gas are preferably used. Preferable examples of the treatment gas are as follows.

### (Oxygen-Based Gas)

Oxygen, ozone, water, carbon monoxide, carbon dioxide, nitrogen monoxide, and nitrogen dioxide

### (Rare Gas)

Argon, xenon, krypton, nitrogen, helium, and neon

### (Fluoride-Based Gas)

Fluorine-carbon compound such as tetrafluorocarbon (CF₄), hexafluorocarbon (C₂F₆), hexafluoropropylene (CF₃CFCF₂) or octafluorocyclobutane (C₄F₈); halogen-carbon compound such as monochlorotrifluorocarbon (CClF₃); and fluorine-sulfur compound such as sulfur hexafluoride (SF₆)

### (Chlorine-Based Gas)

Cl₂, HCl, PCl₃, and BCl₃

### EXAMPLES

### Example 1

An amorphous carbon (diamond-like carbon: DLC) film 6 was set on an earth potential of a vacuum apparatus by the method described in reference to FIG. 2. The DLC film is composed of a material comprising 70% carbon and 30% hydrogen. Nitrogen gas was supplied into the vacuum apparatus and controlled to a pressure of 1 Pa. A DC pulse of −10 kV with pulse width 0.5 μsec and duty ratio 0.5 was applied to a cathode electrode 4 opposed to the earth potential, and the amorphous carbon film was irradiated with the electron beam for 2 hours with an input energy of 0.0013 J/cm². A half of the carbon film as a sample was masked so as not to be irradiated with the electron beam, forming a non-irradiated surface as a comparative example. The remaining half of the carbon film was exposed and irradiated with the electron beam.

The surface roughness Ra of the film after the irradiation with electron beam was 0.1 to 1.0 nm both in the irradiated surface and in the non-irradiated surface without difference. This shows that the minute shape of the surface of the carbon film was not particularly changed.

Each friction coefficient of the irradiated surface and the non-irradiated surface was measured. Concretely the measurement was performed by placing a ball made of SUS 304 with φ10 mm on each of the irradiated surface and the non-irradiated surface, and applying a load of 1N thereon. As a result, the friction coefficient of the electron beam-irradiated surface was 0.11 in contrast to 0.15 in the non-irradiated surface. Namely the friction coefficient could be successfully reduced by the irradiation with the electron beam with the surface shape of the irradiated surface being hardly changed.

Measurement of hardness of the film surfaces was performed by using a nanoindenter. Consequently the hardness of the irradiated surface was 18 GPa in contrast to 16 GPa of the non-irradiated surface, and the hardness was also improved.

### Example 2

A photoresist mask 6 was treated by the method described in reference to FIG. 3. The photoresist (OFPR-800) used has a carbon content of about 50%. The carbon content of the photoresist corresponds to the value after baking. A silicon wafer 6 coated with the photoresist mask was set on an anode electrode 5 in the vacuum apparatus. A half of the photoresist of the wafer was masked with a slide glass to form an electron beam non-irradiated surface. The remaining half was taken as an irradiated surface. The chamber was used as a cathode electrode (earth potential). Argon gas was supplied into the vacuum chamber and controlled at a pressure of 2.0 Pa. A DC pulse voltage of +10.5 kV with pulse width 0.2 μsec (200 nsec) and duty ratio 0.04 was applied to the anode electrode, and the photoresist was irradiated with the electron beam for 10 minutes in a condition of input energy 0.00020 J/cm².

The surface roughness Ra of the film before the irradiation with the electron beam was 8.1 nm, and the surface roughness of the electron beam-irradiated surface was 8.5 nm. Namely only an error level as small as 0.4 nm was observed.

With respect to the non-irradiated surface and the irradiated surface of the photoresist, plasma etching resistance was evaluated. Concretely the etching resistance was evaluated by performing etching in the following condition and measuring the etching quantity.

A photoresist treated with the electron beam and a non-treated photoresist were set on the electrode followed by evacuation. Etching was performed thereto for 35 minutes in a condition of argon gas 1.9 Pa with pulse voltage −13.4 kV, pulse period 5.2 kHz and input power 93 W. After the etching, the etching quantity was measured by a contact type level measurement system.

As a result, the etching rate of the photoresist not treated with the electron beam was 4.1 nm/min in contrast to 2.6 nm/min of the electron beam-treated photoresist. Namely it was confirmed that the plasma etching resistance of the photoresist was improved by the electron beam treatment.

Although specific embodiments of the present invention were described so far, the present invention is never limited by these specific embodiments. The present invention can be carried out with various modifications and alterations without departing from the scope of the accompanying claims.

1. A method for reforming a carbonaceous material comprising 30 atm % or more of carbon atoms and having a surface; said method comprising the steps of:
applying a DC pulse voltage on an electrode set within a chamber to generate an electron beam; and
irradiating said electron beam onto said surface of said carbonaceous material to reform said carbonaceous material,
wherein said DC pulse voltage has a duty ratio of pulse duration per pulse of 0.005 to 5.0%, an input energy of 0.01 J/cm² or less and a pulse half-value width of 10 to 900 nsec.

2. The method of claim 1, wherein said DC pulse voltage has a pulse period of 0.01 to 100 kHz and a pulse voltage of ±0.1 to ±30 kV.

3. The method of claim 1, wherein said carbonaceous material comprises a carbon film.

4. The method of claim 1, wherein said carbonaceous material comprises a low-dielectric constant material.

5. The method of claim 1, wherein an internal pressure in said chamber is 0.1 to 1000 Pa.

6. The method of claim 2, wherein said carbonaceous material comprises a carbon film.

7. The method of claim 2, wherein said carbonaceous material comprises a low-dielectric constant material.

8. The method of claim 2, wherein an internal pressure in said chamber is 0.1 to 1000 Pa.

9. The method of claim 3, wherein an internal pressure in said chamber is 0.1 to 1000 Pa.

10. The method of claim 4, wherein an internal pressure in said chamber is 0.1 to 1000 Pa.
